(19) United States
(12) Patent Application Publication
(10) Pub. No.: US 2010/0322989 A1
(43) Pub. Date: Dec. 23, 2010

(54) HYDROPHILIC SKIN CLEANING CLOTH

(76) Inventor: Juergen Anton Martin, Thayngen (CH)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/866,949

(22) PCT Filed: Feb. 12, 2009
(86) PCT No.: PCT/EP09/51651

§ 371 Date: Aug. 10, 2010

(30) Foreign Application Priority Data
Feb. 13, 2008 (CH) 00202/08

(51) Int. Cl. A61K 8/02; A61K 36/00; A61P 9/00; A61P 43/00; A61P 29/00; A61P 31/00; A61P 11/02; A61P 17/02; A61Q 19/04; A61K 31/01

(52) U.S. Cl. 424/402; 424/725; 424/59; 514/762

(57) ABSTRACT

A preferably single-ply, simply and inexpensively manufacturable hydrophilic skin care tissue for disposable use comprising a textile surface (1) of microfibres is described. The textile surface (1) has run through a splitting process and comprises a plurality of barbs (14) and accordingly has an abrasiveness with which a micro-dermal abrasion of the facial skin of a user may be carried out. A defined quantity of at least one active ingredient (2) is stored in a mechanically held manner in the textile surface (1) on bows, microfibres and/or grid intermediate spaces (11). The at least one active ingredient is released from the inside of the textile surface (1) by way of the mechanical deformation, and by way of this gets to the pre-treated skin of the user.

### TECHNICAL FIELD

The present invention describes a hydrophilic, single-ply skin care tissue, comprising a textile surface with a grid structure and grid intermediate spaces, with an abrasive effect, which permits a mechanical micro-dermal abrasion, and is manufactured of a mixture of hydrophilic and hydrophobic material.

### STATE OF THE ART

Cleaning tissues for the skin which, with the help of water or other cleaning fluids to which active ingredients are added, have above all been known for some time for cleaning the facial skin

For some time now, cleaning tissues which permit a so-called superficial peeling of the uppermost subcorneous layer of the skin only by way of the application of mechanical means have been known. This superficial peeling is called micro-dermal abrasion, wherein the uppermost subcorneous layer of the skin is freed of dead cells. The used cleaning tissues mostly consist of fabric, which has been woven from individual microfibres with fibre diameters in the micrometer region. The fabric of the cleaning tissues, which is manufactured of microfibers, is characterised by a large surface area, by which means fluid may be stored extremely well and the tissue has a high absorption capability, which corresponds to a high water absorption coefficient. In the past, the surface area could be increased even more by way of smaller and smaller fibre diameters, by which means the absorption capability could be increased yet again. The optimisation of the mixture ratio of hydrophilic and hydrophobic material additionally optimised the water absorption coefficient.

For the micro-dermal abrasion to be possible with a cleaning tissue of microfibres, the fabric after the weaving or knitting is split in a so-called splitting process, by which means the outer layers of the individual microfibres are roughened, and barb-like structures arise, which increase the abrasiveness of the tissue, by which means skin layers may be removed. Moreover, the water absorption capability is also improved by the splitting process, since water molecules are retained within the tissue by way of barbs. The degree of the splitting process, apart from the fluffiness of the fabric and the abrasiveness which this entails, also influences the water absorption capability.

Usually, after the peeling, cosmetic or pharmaceutical active ingredients may be deposited onto the skin for the care and protection of the skin, wherein this supply of active ingredients is effected temporally after the cleaning and separately from the cleaning

### DESCRIPTION OF THE INVENTION

It is the object of the present invention to provide a skin care tissue, with which a mechanical a micro-dermal abrasion of the uppermost subcorneous layer of the skin may be combined with a local active ingredient application, which with regard to time, is effected directly subsequent to this, wherein the degree of the micro-dermal abrasion of the skin and the quantity and the type of the applicable active ingredients is set by the skin care tissue.

The skin care tissue according to the invention is simple to use and ensures the implementation of a peeling, wherein active ingredients are deposited onto the skin in the same working step, wherein only the one skin care tissue is applied.

In a special embodiment, an inexpensively manufacturable, light skin care tissue is claimed, which is manufactured as a disposable product for disposable use.

### BRIEF DESCRIPTION OF THE DRAWINGS

A preferred embodiment example of the subject matter of the invention is described hereinafter in combination with the accompanying drawings.

FIG. 1 shows a schematic view of a somewhat two-dimensional fabric of microfibres, whilst

FIG. 1b shows a detailed view of a cut-out of the 2d-fabric of FIG. 1a, wherein the grid structure and the barbs are clear.

FIG. 1c shows a fabric according to FIG. 1b, which is moistened with active ingredients, essentially within the 2d-fabric.

FIG. 2a shows a schematic view of a three-dimensional fabric of microfibres, said fabric comprising bows, whilst

FIG. 2b shows a detailed view of the tissue of FIG. 2a, wherein barbs caused by splitting are clear on the grid structure and on the bows.

FIG. 2c shows a tissue according to FIG. 2b, which is wetted with a share of at least one active ingredient

### DESCRIPTION

The basis of the present skin care tissue according to the invention is formed by a textile surface 1 which is woven or knitted and which is hydrophilic on account of the applied materials and/or on account of the applied manufacturing method, and may absorb and retain water or fluids in large quantities. These requirements are fulfilled by a textile surface 1 of microfibres, which comprises a grid structure 10 with empty grid intermediate spaces 11. The applied microfibres usually have a diameter in the region of smaller than 50 μm.

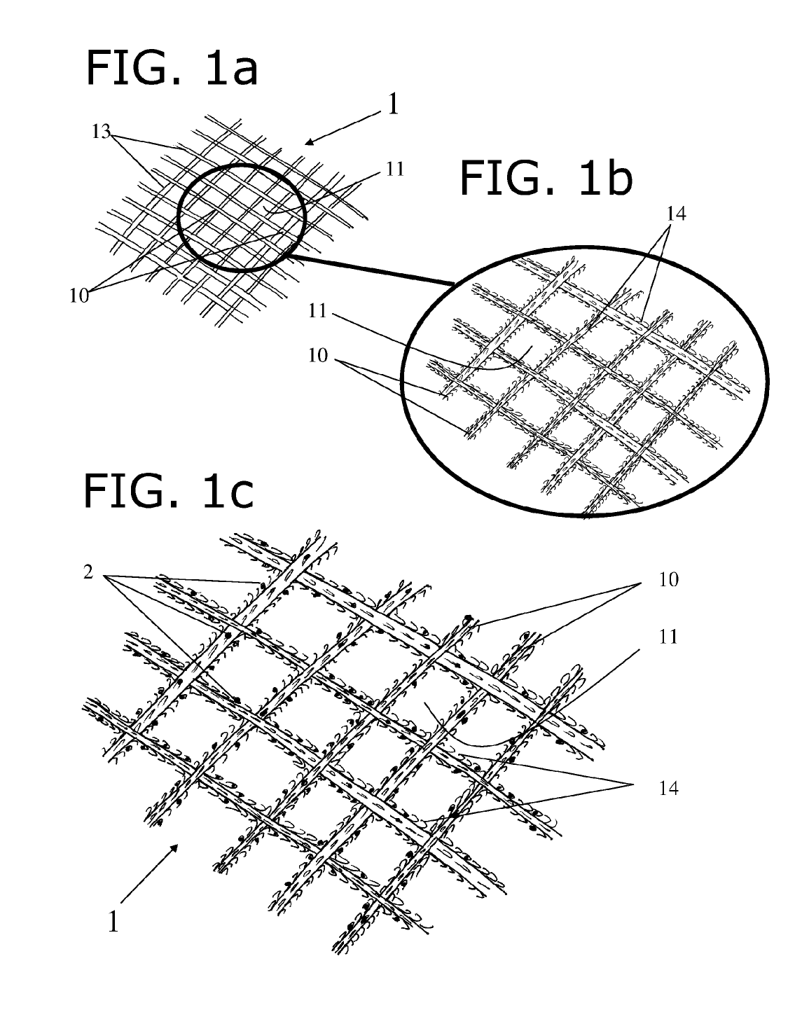

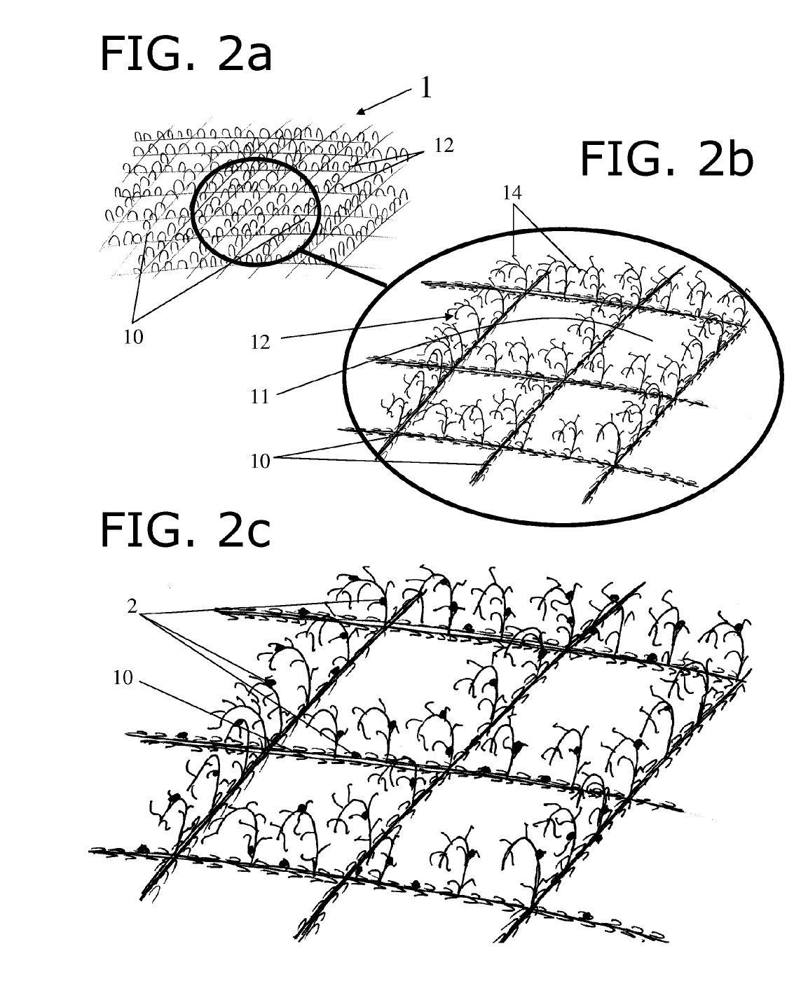

The applied weaving type and the microscopic grid structure 10 of the textile surface 1 of the skin care tissue are variable. One example of a somewhat two-dimensional, single-ply grid structure 10 of the textile surface is shown in FIG. 1a, whilst a somewhat three-dimensional grid structure 10 of the textile surface 1 with bows 12 pointing away from the plane of the grid structure 10 in an approximately perpendicular manner, is shown in FIG. 2a.

The skin care tissue consisting of microfibres has a high water absorption coefficient. The water absorption coefficient is a measure for the water absorption capability and indicates the amount of fluid which may be absorbed by a standard surface per time unit. The fluid, by way of capillary or absorptive forces, is absorbed by the textile surface 1 and is held in the grid structure 10. Thereby, the molecules of the fluid may directly settle in the grid intermediate spaces 11 and/or on the hydrophilic microfibres 13.

In order to achieve the required abrasiveness for the skin care tissue according to the invention and to achieve an as high as possible water absorption coefficient, the textile surface 1 after the weaving or kitting, is roughened in a splitting process, by which means the surface area of the textile surface may yet be considerably increased. The splitting process is usually carried out by an alkaline bath of the woven or knitted textile surface 1, by which means the casing which surrounds the individual microfibres 13, is partly dissolved in a fibred manner. The splitting process leads to the formation of barbs 14, as are to be recognised in the Figures lb and 2b. By way of these barbs 14, apart from the surfaces area and the water absorption capacity which this entails, the abrasiveness of the textile surface 1 is also increased. The abrasiveness of the textile surface 1 may be influenced by the degree of the splitting process, which in turn determines the peeling characteristics. The number and the length of the individual barbs 14 are designed in accordance with the degree of the splitting process.

The skin care tissue according to the invention is preferably manufactured of a material mixture of polyamide and polyester. Trials has found that a polyamide content of greater than 10%, preferably greater that 25% must be used for an adequately hydrophilic surface 1. Polyamide is hydrophilic, which is why the water absorption capability is correlated to the polyamide content. Very good results have been achieved with a polyamide content of 40% and a polyester content of 60%. Apart from the hydrophobic polyester, other hydrophobic materials may also be applied. The textile surface 1 after the weaving or knitting is treated further in a splitting process until a piece of the textile surface 1 with a given intrinsic weight may absorb a water quantity of the magnitude of a multiple of the intrinsic weight of the textile surface 1. A skin care product whose textile surface 1 may absorb twofold to threefold its intrinsic weight of water is particularly preferably.

By way of the splitting process, a hydrophilic skin care tissue with an abrasive effect arises, which has a surface weight, a weight per standard surface of one square meter, of a few hundred grams per square metre. Since the skin care tissue according to the invention is preferably applied as a disposable product for disposable use, textile surfaces 1 with a surface weight of less than two hundred grams per square meter are envisaged for reasons of cost.

After the textile surface 1 of the skin care tissue has been refined in a spitting process and the barbs 14 project from the microfibres 13 and partly project into the grid intermediate spaces, as may be recognised in the Figures lb and 2b, at least one active ingredient 2 is introduced into the textile surface 1. These active ingredients may be designed in a water-soluble or fat-soluble manner and may originate from the class of pharmaceutical or cosmetic active ingredients. The active ingredient 2 is held mechanically within the grid structure 10 on the bows 12, the microfibres 13 per se or the branches of the barbs 14. A defined mass of a reproducible magnitude of the active ingredient 2 is stored within the textile surface 1.

Preferably, a mass of the active ingredient is within the textile surface 1, which corresponds to about 2.5% to 7.5% of the intrinsic weight of the textile surface 1, in particular a mass of the active ingredient is preferred, which corresponds to about 2.5% to 5% of the intrinsic weight of the textile surface 1. Trials have shown that depending on the field of application, a skin care tissue with a surface weight of 200 g/m² with a defined quantity of active ingredient 2 of 5 to 10 g/m² provides good results for the disposable use. With such a charge of the textile surface 1, one may deposit an adequate amount of active ingredient 2 onto the skin of the user.

The active ingredients are introduced onto the textile surface 1 in different manners. For example, the active ingredient may be dissolved in water or a solvent or may be present in a water/active ingredient emulsion in a steep bath, through which the textile surface 1 is pulled. Individual particles of the active ingredient 2 hook in the grid structure 10 with this process, and are mechanically attached. This attachment of the active ingredient 2 here is indicated as being mechanically held within the textile surface 1. Excess water may be then removed subsequently by way of evaporation.

A defined constant mass of at least one active ingredient 2, as is known to the average man skilled in the art, may be deposited by way of a targeted vapour deposition or spray deposition of a solution of the active ingredient 2 in water or of a water/active ingredient emulsion onto the textile surface 1.

If the active ingredient 2 is present in powdered form or in a micro-encapsulated manner, an atomisation or an impingement of the textile surface 1 with the active ingredient 2 may be carried out, wherein a controllable quantity of active ingredient remains attached on the textile surface 1. If the active ingredient 2 is deposited atomised or impinged onto the textile surface 1 in powder form or in a micro-encapsulated manner, then a thermal method may be applied in a supporting manner for fixation, by which means the active ingredient 2 is thermally bonded to the polyamide.

The textile surface 1 which is impinged with the active ingredient 2, may be dried after the submersion bath, the vapour deposition or the spray deposition, by way of a thermal method, wherein water molecules evaporate out of the textile surface 1 and the pure active ingredient 2 remains.

The incorporation of a mass of an active ingredient 2 into the textile surface 1 may also be achieved by way of a powder coating. Thereby, it is possible to electrostatically charge the active ingredient 2 present in powder form and/or the textile surface 1, and move the textile surface 1 at a small distance over this powder. Thereby, the active ingredient 2 is accelerated in the direction of the textile surface 1 and penetrates deeply into the textile surface 1 ramified with barbs 14 and there is held mechanically within the textile surface 1. A subsequent thermal process, here too, may increase the permanent fixation of the active ingredient 2.

Examples of possible active ingredients 2 are vegetable oils, essential and/or mineral oils, but also water-soluble pharmaceutics active ingredients 2 which act in an astringent, circulation- encouraging, revitalising, inflammation-reducing, disinfecting or tightening manner. For example, apart from sun lotion for UV-protection of the skin, cosmetic active ingredients 2 may be applied, for example tanning agents or bleaching agents for the targeted tanning of the skin. Herbal extracts for calming the skin or further active ingredients 2 which act in an antiseptic and/or decongestant and/or wound-healing and/or slightly cooling and/or itch-relieving manner, are of interest here.

The user uses the skin care tissue essentially as a normal cleaning tissue of the state of the art, which is moistened with water or another fluid and is mostly moved over the skin with circular movements. Due to the abrasiveness of the skin care tissue, excess dead skin cells of the uppermost skin layers are removed. The mechanically held active ingredients 2 are released out of the textile surface 12 from the ramifications of the barbs 14 by way of the mechanical deformation of the textile surface 1 on movement of the skin care tissue, by which means the active ingredients 2 get to the skin and may be deposited there in a local manner.

If the active ingredient 2 is present in a water-insoluble and micro-encapsulated manner, then the active ingredient 2 does not necessarily dissolve in water, so that the particles of the active ingredient 2 need more time, until these are mechanically released out of the textile surface 1 from the grid structure 10, from the grid intermediate spaces 11, from the bows 12, from the microfibres 13 or barbs 14, and may get to the skin. Since a defined mass of the active ingredient 2 is mechanically held in the textile surface 1, the correct and reproducible dosing of the active ingredients 2 is always given and an overdose is ruled out.

The skin care tissue according to the invention of the hydrophilic textile surface 1 with an abrasive effect and which provided with active ingredients 2, absorbs moisture on use, by which means the active ingredients 2 may be released from the inside of the tissue 1. After the active ingredients have been released by way of the water addition and movement of the skin care tissue, the active ingredients may be introduced onto the skin and in deeper lying skin layers on account of the preceding peeling treatment. A reproducible dosing and a local deposition of the active ingredients 2 onto the skin is effected on account of the defined quantity of the mechanically stored active ingredient. Apart from the skin care tissue, no further peeling devices or active-ingredient-containing lotions or creams are required, which is why a cosmetic and/or pharmaceutical treatment is possible alone with the skin care tissue according to the invention.

In a further inventive embodiment of the skin care tissue, the active ingredient 2 is stored in the fabric 1 in the form of nanoparticules with sizes of a few hundred nanometres up to a few tens nanometres.

The preferred use of the skin care tissue for disposable use prevents dirt particles, dead skin cells or microorganisms which are already preset in skin care tissue, which have already been used several times, from coming into contact with the skin.

Microfibres are today still extremely expensive and the use of these as disposable tissues contradicts normal commercial considerations. The cost factor may be reduced by way of the use of single-ply tissues with a low weight per area. On the other hand the reusability is practically ruled out by way of the combination of micro-dermal abrasion and deposition of active ingredients 2 which is of interest here, since this necessitates a washing procedure which removes the active ingredients.

### LIST OF REFERENCE NUMERALS

1 textile surface
10 grid structure
11 grid intermediate spaces
12 bows
13 micro-fibres
14 barbs
2 active ingredients

1. A hydrophilic, single-ply skin care tissue comprising a textile surface with a grid structure and grid intermediate spaces, with an abrasive effect, which permits a mechanical, micro-dermal abrasion and is manufactured of a mixture of hydrophilic and hydrophobic material, characterised in that the textile surface is charged with a defined quantity of at least one active ingredient which is held mechanically within the textile surface, wherein the active ingredient by way of the fluid supply and mechanical deformation of the textile surface, may be released from the inside of the textile surface and by way of this may be deposited locally onto the skin.

2. A skin care tissue according to claim 1, characterised in that the textile surface consists of microfibres which have a diameter of less than 50 μm, are roughened in a splitting process and comprise a multitude of barbs.

3. A skin care tissue according to claim 1, characterised in that the textile surface is manufactured from a hydrophilic polyamide/polyester mixture with a polyamide share of at least 10%.

4. A skin care tissue according to claim 1, characterised in that the active ingredient is water-soluble and is dissolved with water by way of wetting the skin care tissue.

5. A skin care tissue according to claim 1, characterised in that the active ingredient is fat-soluble.

6. A skin care tissue according to claim 5, characterised in that the at least one active ingredient comprises vegetable oils and/or essential oils and/or mineral oils.

7. A skin care tissue according to claim 1, characterised in that the at least one active ingredient includes pharmaceutical active ingredients which act on the skin in an astringent, circulation-encouraging, revitalising, inflammation-reducing, disinfecting, decongestant, wound-healing, cooling, itch-relieving and/or tightening manner.

8. A skin care tissue according to claim 1, characterised in that the at least one active ingredient is a tanning agent or a bleaching agent for the targeted tanning of the skin.

9. A skin care tissue according to claim 1, characterised in that the at least one active ingredient includes herbal extracts.

10. A skin care tissue according to claim 1, characterised in that the active ingredient is sprayed on, vapour deposited and/or is introduced into the textile surface by way of pulling through a submersion bath.

11. A skin care tissue according to claim 1, characterised in that the textile surface may absorb roughly at least approximately double to threefold its intrinsic weight of water.

12. A skin care tissue according to claim 1, characterised in that the textile surface of the skin care tissue has a surface weight of less than two hundred grams per square meter.

13. A skin care tissue according to claim 12, characterised in that the skin care tissue is designed as a disposable product for disposable use.

14. A skin care tissue according to claim 1, characterised in that the share of the at least one active ingredient within the textile surface corresponds to less than 10% of the mass of the textile surface.

15. A skin care tissue according to claim 14, characterised in that the share of the at least one active ingredient which is deposited onto the textile surface corresponds to about 2% to 7.5% of the mass of the textile surface.
